(19) United States
(12) Patent Application Publication
(10) Pub. No.: US 2020/0046969 A1
(43) Pub. Date: Feb. 13, 2020

(54) RF VAGINAL REJUVENATION

(71) Applicant: Home Skinovations Ltd., Yokneam (IL)

(72) Inventors: BenZion Levi, Habonim (IL); Eyal Agassi, Karmiel (IL)

(73) Assignee: Home Skinovations Ltd., Yokneam (IL)

(21) Appl. No.: 16/059,188

(22) Filed: Aug. 9, 2018

(51) Int. Cl. A61N 1/32; A61B 18/14

(52) U.S. Cl. CPC A61N 1/328; A61F 2007/0087; A61B 18/1485

(57) ABSTRACT

A device includes a shaft made of an electrically insulating material, RF electrodes axially spaced one from another by insulating gaps along an axial length of the shaft, a handle coupled to the shaft, and RF and electrical components coupled to the handle and in electrical communication with the RF electrodes. A peripheral shape of the shaft is triangular with rounded corners.

### FIELD OF THE INVENTION

The present invention generally relates to vaginal rejuvenation, and more particularly to an RF (radio-frequency) device and method for vaginal rejuvenation.

### BACKGROUND OF THE INVENTION

Vaginal tissues and the pelvic floor can be weakened and become loose due to multiple child births, aging, or genetic disorders. Generally the inner and outer dimensions of the vagina increase with age.

RF vaginal rejuvenation uses RF energy to heat collagen in the upper layer of the vaginal tissue to promote new collagen regeneration, which induces tightening of the vaginal walls.

One of the problems associated with RF vaginal rejuvenation is that RF electrodes must be in close contact with the vagina wall so that RF impedance closely matches the tissue. If the contact between the electrode and the vaginal wall is poor, the RF energy can reflect off the vaginal wall, which can significantly degrade performance of the device and cause discomfort or injury to the patient.

### SUMMARY OF THE INVENTION

The present invention seeks to provide a novel and improved RF device and method for vaginal rejuvenation, as described in detail below. The device has a novel shape which improves close contact of the RF electrodes with the vaginal wall to overcome the problems of the prior art.

There is thus provided in accordance with a non-limiting embodiment of the present invention a device including a shaft made of an electrically insulating material, RF electrodes axially spaced one from another by insulating gaps along an axial length of the shaft, a handle coupled to the shaft, and RF and electrical components coupled to the handle and in electrical communication with the RF electrodes, wherein a peripheral shape of the shaft is triangular with rounded corners. The peripheral shape of the shaft may be a Reuleaux triangle with rounded corners.

In accordance with a non-limiting embodiment of the invention the RF electrodes do not extend fully about an entire periphery of the shaft.

In accordance with a non-limiting embodiment of the invention the RF generator and electrical components are in electrical communication with the RF electrodes via a flexible or non-flexible printed circuit board, or flexible cables.

In accordance with a non-limiting embodiment of the invention a thermal sensor but not limited to one sensor, is in thermal contact with one of the RF electrodes. The thermal sensor may be in thermal contact with one of the RF electrodes via a thermal pad or thermal grease.

In accordance with a non-limiting embodiment of the invention one or more distal pairs of the RF electrodes are visually different than the rest of the RF electrodes.

In accordance with a non-limiting embodiment of the invention one or more distal pairs of the RF electrodes are made of or coated with a different metal conductive coating than the rest of the RF electrodes.

In accordance with a non-limiting embodiment of the invention a method is provided including inserting the device into a vagina and sequentially applying RF energy between pairs of the RF electrodes along a length of the shaft while the shaft remains stationary, wherein application of the RF energy causes remodeling of collagen in a wall of the vagina. Alternatively, a method is provided for treating the appearance of the vulva. The pairs of the RF electrodes, coated with a different coating, are applied on the external part of the vulva by moving them all over the treated area including the labia, which causes remodeling of collagen and improvement of vulva appearance.

### BRIEF DESCRIPTION OF THE DRAWINGS

The present invention will be understood and appreciated more fully from the following detailed description, taken in conjunction with the drawings in which:

FIG. 1 is a simplified perspective illustration of an RF vaginal rejuvenation device, in accordance with a non-limiting embodiment of the present invention;

FIG. 2 is a simplified side-view illustration of the RF vaginal rejuvenation device;

FIG. 3 is a simplified end-view illustration of the RF vaginal rejuvenation device;

FIG. 4 is a simplified perspective illustration of inner components of the RF vaginal rejuvenation device;

FIG. 5 is a simplified graphical illustration of sequential RF energy delivery between neighboring electrodes along the length of the RF vaginal rejuvenation device, in accordance with a non-limiting embodiment of the present invention; and

FIG. 6 is a simplified graphical illustration of a timing sequence of RF energy delivery with the RF vaginal rejuvenation device in accordance with a non-limiting embodiment of the present invention.

### DETAILED DESCRIPTION OF EMBODIMENTS

Reference is now made to FIGS. 1-3, which illustrate an RF vaginal rejuvenation device 10, constructed and operative in accordance with a non-limiting embodiment of the present invention.

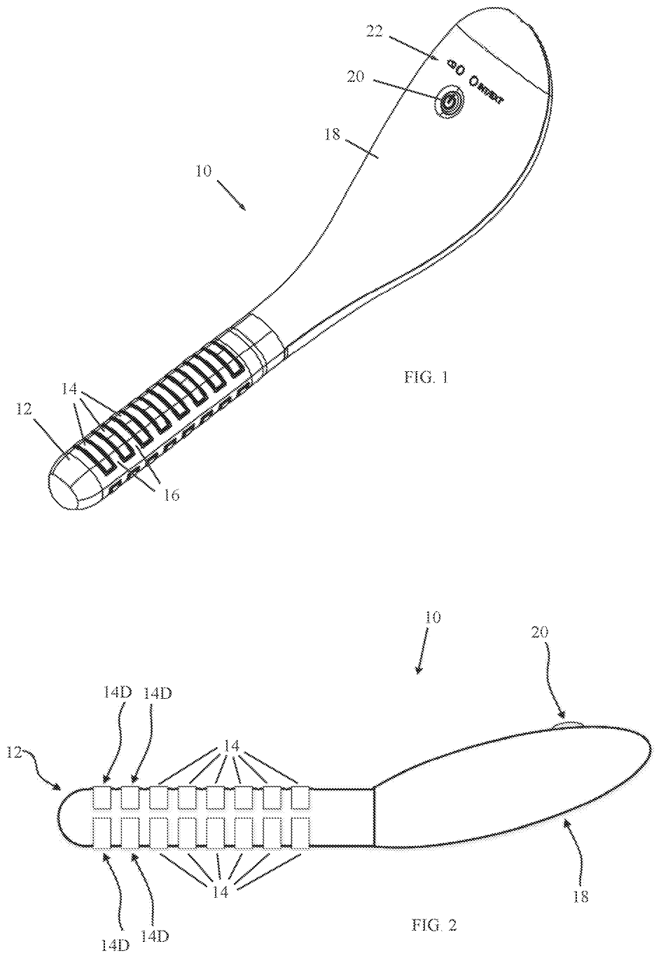

The device 10 includes a shaft 12, which may be made of an electrically insulating material, such as but not limited to, thermoplastic polyurethane (TPU). RF electrodes 14 are axially spaced one from another along an axial length of shaft 12 with axial insulating gaps 16 between them (insulating gaps because the gaps are portions of the shaft 12). The RF electrodes 14 may be partially peripheral, as in the illustrated embodiment of FIG. 2, in which there are upper and lower electrodes 14A and 14B, respectively. Alternatively, the RF electrodes 14 may extend fully about the entire periphery of the shaft 12.

A handle 18 may be coupled to shaft 12. The handle 18 may include various operating controls 20 (such as, but not limited to, on/off buttons, touch screens, operating mode selectors and more), and indicators 22 (such as, but not limited to, display lights, display screens and more).

Reference is now made to FIG. 4. The inner parts of the handle 18 may include various components for operating the device 10, such as, but not limited to, an RF generator transformer 24, a battery 26 and control circuitry components 28 for controlling the RF energy delivery. The components may be in electrical communication with the electrodes 14 via flexible printed circuit boards 30 or other suitable connectors. The components may be alternatively housed in the shaft instead of the handle or they may be external to both the handle and the shaft.

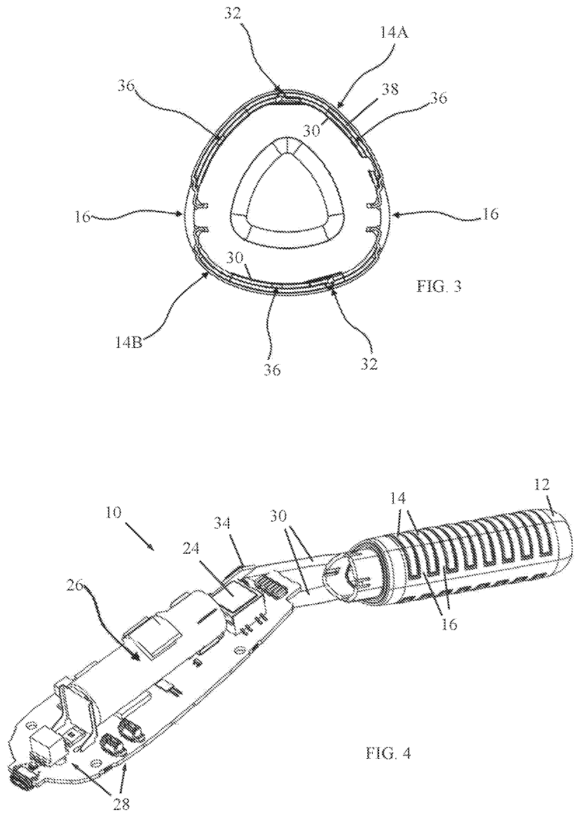

Reference is now made particularly to FIG. 3. The electrical connection between each electrode 14A or 14B and the connector 34 may be achieved with an electrical connector 32, such as a C-clip connector, which is in electrical contact with the electrode and may be mounted on the flexible printed circuit board 30, which is in electrical contact, via the connector 34 with the components for operating the device 10. A thermal sensor 36 (such as, but not limited to, a thermistor which may have a negative temperature coefficient (NTC) or positive temperature coefficient (PTC)) may be mounted on flexible printed circuit board 30 and may be in thermal contact with at least one of the RF electrodes, such as by means of a thermal pad or thermal grease 38. The thermal sensor 36 may be used to sense the temperature of the vaginal wall or the vulva during operation and be used in a control loop to regulate the RF energy delivery in accordance with the sensed temperature to avoid charring or other thermal damage.

In one embodiment, thermal sensor 36 may be provided for each one of the RF electrodes 14 so that the temperature of the vaginal wall at each one of the RF electrodes 14 is monitored. If the temperature of the vaginal wall or the vulva in contact with one of the electrodes has reached the desired temperature, then the power to that electrode can be shut off. This arrangement of the thermal sensors 36 facilitates reaching a uniform steady-state temperature at every place along the vaginal wall or the vulva surface.

As seen in FIG. 3, in accordance with a non-limiting embodiment of the present invention, the peripheral shape of shaft 12 is not circular but instead triangular with rounded corners. In one embodiment, the peripheral shape of shaft 12 is a Reuleaux triangle with rounded corners. A Reuleaux triangle without rounded corners is a shape formed from the intersection of three circular disks, each having its center on the boundary of the other two. The boundary of the Reuleaux triangle is a curve of constant width. (The shape of the rotor of a Wankel engine is similar, but not identical, to the Reuleaux triangle.) It is believed the peripheral triangular shape of shaft 12 with rounded corners better conforms to the vulva surface and to the inner shape of the vagina and enhances contact between the electrodes and the vaginal wall, thereby increasing efficiency of the RF energy delivery and significantly decreasing chances of pain to the user.

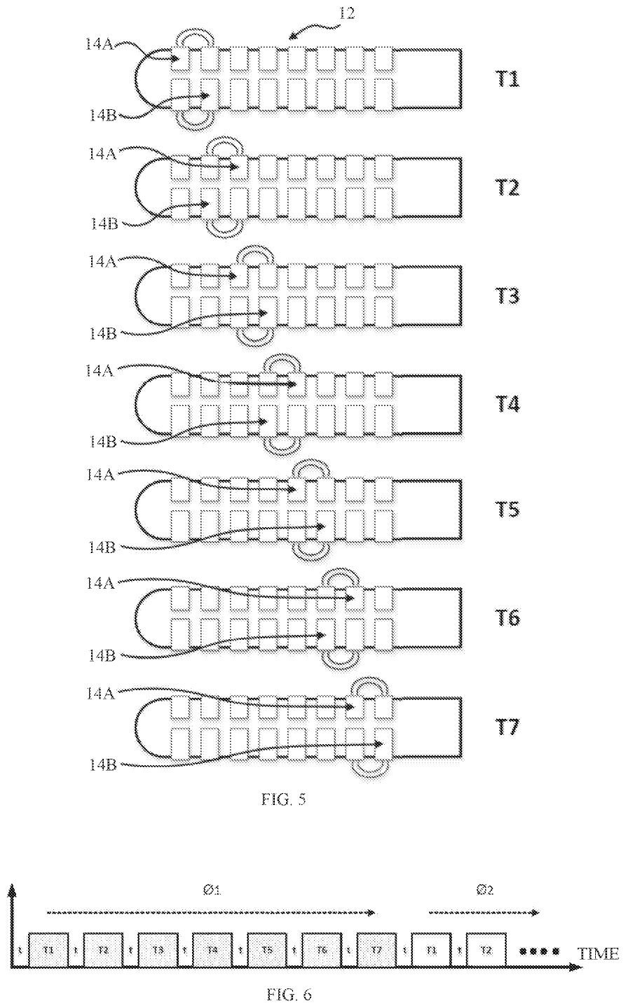

Reference is now made to FIGS. 5 and 6, which illustrate one method of sequential RF energy delivery between neighboring electrodes 14 along the length of the shaft 12 of the RF vaginal rejuvenation device 10. For time duration=T1 (e.g., without limitation, 100 ms or in the range of 70-150 ms), RF energy may be delivered between the two most distal electrodes. After a pause of no RF delivery for a duration of time=t (e.g., without limitation, 10 ms or in the range of 7-15 ms), the RF energy is delivered during a time duration=T2 between the next pair of electrodes and so forth over the length of the shaft 12. In this manner, RF energy is delivered to the entire vaginal wall while shaft 12 remains stationary.

As see in FIG. 6, different operating phases may be used with different durations, frequencies, amplitudes and other parameters that define the RF energy being delivered. For example, without limitation, RF energy may be delivered at different RF frequencies, such as 0.5-10 MHz with power from 0.1-12 W.

The RF vaginal rejuvenation device 10 may also be used for external treatment of the vulva. In accordance with a non-limiting embodiment of the present invention, one or more distal pairs of RF electrodes (designated 14D in FIG. 2), may be visually different than the rest of the RF electrodes. For example, the one or more distal pairs of RF electrodes 14D may be made of or coated with a different metal (e.g., made of silver or silver coated, as opposed to the rest of the RF electrodes, which may be gold coated or made of gold). This is advantageous as an indication to the user as to which electrodes should be used for external RF treatment of the vulva as opposed to the rest of the RF electrodes which may be for internal use only (RF electrodes 14D are for internal and external use). The triangular peripheral shape of the shaft is particular advantageous for external RF treatment of the vulva and especially the genital labia.

What is claimed is:

1. A device comprising:
a shaft made of an electrically insulating material;
RF electrodes axially spaced one from another by insulating gaps along an axial length of said shaft;
a handle coupled to said shaft; and
RF and electrical components coupled to said handle and in electrical communication with said RF electrodes;
wherein a peripheral shape of said shaft is triangular with rounded corners.

2. The device according to claim 1, wherein the peripheral shape of said shaft is a Reuleaux triangle with rounded corners.

3. The device according to claim 1, wherein said RF electrodes do not extend fully about an entire periphery of said shaft.

4. The device according to claim 1, wherein said RF and electrical components are in electrical communication with said RF electrodes via flexible cables.

5. The device according to claim 1, wherein a thermal sensor is in thermal contact with one of said RF electrodes.

6. The device according to claim 1, wherein a thermal sensor is in thermal contact with one of said RF electrodes via a thermal pad or a thermal grease.

7. The device according to claim 1, wherein one or more distal pairs of said RF electrodes are visually different than the rest of the RF electrodes.

8. The device according to claim 1, wherein one or more distal pairs of said RF electrodes are made of or coated with a different metal than the rest of the RF electrodes.

9. A method comprising inserting the device of claim 1 into a vagina and sequentially applying RF energy between pairs of said RF electrodes along a length of said shaft while said shaft remains stationary, wherein application of the RF energy causes remodeling of collagen in a wall of the vagina.
